(19) United States
(12) Patent Application Publication
(10) Pub. No.: US 2024/0197530 A1
(43) Pub. Date: Jun. 20, 2024

(54) REDUCING VIBRATION IN A VITRECTOR

(71) Applicant: JOHNSON & JOHNSON SURGICAL VISION, INC., Irvine, CA (US)

(72) Inventors: Alexander Shechtman, Haifa (IL); Ilya Sitnitsky, Nahariya (IL); Assaf Govari, Haifa (IL); Stanislav Katzir, Hadera (IL); Elad Avraham Diukman, Haifa (IL)

(21) Appl. No.: 18/084,674

(22) Filed: Dec. 20, 2022

(51) Int. Cl. A61F 9/007

(52) U.S. Cl. CPC A61F 9/00763; A61B 2017/00398

(57) ABSTRACT

Surgical apparatus, including a needle configured to be inserted into an organ of a human subject, a counterweight, and a needle driver. The needle driver includes a cylindrical cam configured to rotate about a cam axis and having a curved circumferential groove extending around a radial surface of the cam. There is a motor coupled to rotate the cylindrical cam about the cam axis. A first follower is mounted to travel in the groove at a first azimuthal location and is coupled to the needle so as to cause the needle to oscillate at a predefined frequency parallel to the cam axis as the cam rotates. A second follower is coupled to the counterweight and is mounted to travel in the groove at a second azimuthal location selected so as to cause the counterweight to oscillate parallel to the cam axis in antiphase to the needle.

### FIELD OF THE DISCLOSURE

This disclosure relates generally to vitrectomy, and specifically to tools used for vitrectomy.

### BACKGROUND

Vitrectomy is surgery that removes vitreous humor from an eye, and the surgery is performed using a vitrector, which comprises a hollow needle through which the vitreous humor is aspirated. Following are summaries of references that may apply to vitrectors.

U.S. Patent Application 2021/0077298 to Reyes et al., describes a vitrectomy probe having vibration dampening and attenuating. The probe includes minimizing the degree of flow restriction through a housing that facilitates cutter reciprocation by way of air pressure.

U.S. Pat. No. 10,758,411 to Dean et al., describes a handheld reciprocating surgical tool having an inertial damper to counteract the momentum of a diaphragm assembly of the reciprocating surgical tool. A momentum of the inertial damper may be tuned such that the momentum of the inertial damper is comparable in magnitude and opposite in direction to a momentum of the diaphragm assembly.

U.S. Pat. No. 10,251,782 to Farley describes a probe having a body arranged for grasping by a surgeon and a cutting element extending distally from the body. The probe has an actuating element including a first coil secured within the body, a first magnet operatively secured to an inner member, and a second magnet that is not secured to the inner member, the second magnet being positioned and arranged to move in an opposite direction of the first magnet upon application of a voltage to the first coil.

U.S. Pat. No. 9,750,639 to Barnes et al., describes an electric vitrectomy handpiece. The handpiece includes a motor, a clutch mechanism, an oscillating drive mechanism, a cutting tip and a handle. The motor is attached to the clutch, and the clutch is attached to the oscillating drive mechanism.

U.S. Pat. No. 9,402,766 to Akahoshi et al., describes a phacoemulsification needle that is configured to cause eccentric or wobble-type motion by being formed to distribute the mass of the needle non-uniformly.

### BRIEF DESCRIPTION OF THE DRAWINGS

The present disclosure will be understood from the following detailed description, taken in conjunction with the drawings in which:

FIG. 1 is a perspective view of a vitrector;

FIG. 2 is a cross-section of a part of the vitrector;

FIG. 3 is a cross-section of selected components of the vitrector; and

FIGS. 4A and 4B are views of a cam of the vitrector.

### DESCRIPTION OF EXAMPLES

### Overview

A vitrector is a surgical tool used for removing vitreous humor from an eye, and comprises a hollow cylindrical needle through which the vitreous humor is aspirated. The needle is enclosed in a hollow cylindrical tubular casing, closed at its distal end, and the casing has a window in its side, close to the distal end, via which the vitreous humor transfers to the needle. During operation of the vitrector, the casing with its enclosed needle is inserted into the eye, and the needle is oscillated longitudinally, along the needle's axis of symmetry, within the casing. The oscillations cause the distal tip of the needle to cut the vitreous humor as the tip traverses the casing window; it is the cut portions of the vitreous humor that are aspirated.

The needle is connected by a coupling to a cam follower, and the cam follower is constrained to move longitudinally while travelling in a groove of a cylindrical cam. A motor within the vitrector rotates the cam, causing the cam follower, the coupling, and the needle to oscillate longitudinally. However, the oscillations generate vibrations and noise, both of which may adversely affect a physician's ability to precisely and comfortably operate the vitrector. Surgical procedures within the eye are delicate, so that precise operation of the vitrector is important.

Embodiments of the present disclosure reduce the vibrations and the noise generated by adding a countervailing mass, a counterweight, to the vitrector. The counterweight is connected to a second cam follower which is also constrained to move longitudinally while travelling in the groove of the cylindrical cam. Thus, when the vitrector motor operates, the counterweight also oscillates longitudinally. However, the second cam follower is located in the groove of the cam so that the counterweight oscillations, while being in synchronism with the needle oscillations, are in antiphase to the oscillations of the needle.

The addition of the counterweight, and operating it to oscillate in antiphase to the oscillations of the needle, considerably reduces the vibrations and noise of the vibrating needle.

### System Description

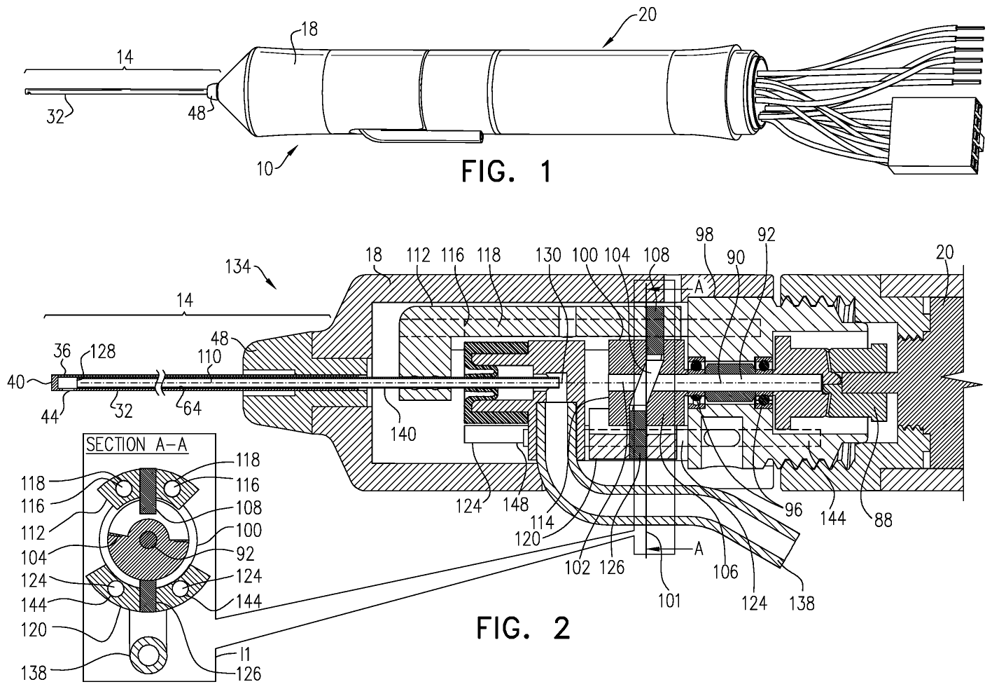

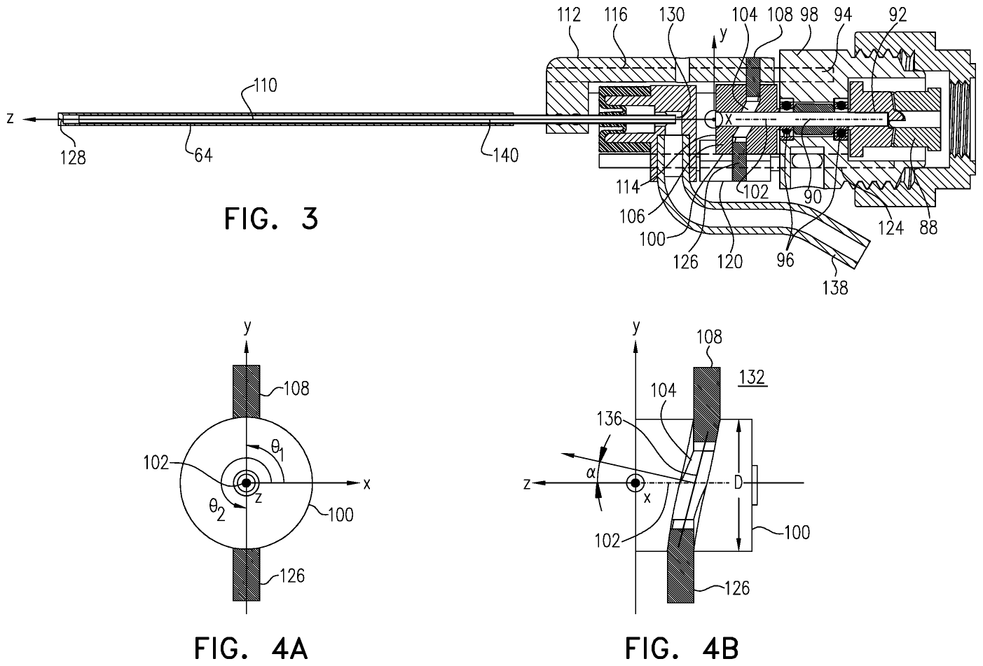

FIGS. 1, 2, 3 and 4A and 4B are schematic diagrams of a vitrector 10. FIG. 1 is a perspective view of the vitrector, FIG. 2 is a cross-section of a part of the vitrector, FIG. 3 is a cross-section of selected components of the vitrector, and FIGS. 4A and 4B are views of a cam of the vitrector. As shown in FIG. 1, vitrector 10 is comprised of a needle portion 14 and a vitrector body 18. A vitrector motor 20 is located within a proximal section of body 18, and a casing coupling 48, described in more detail below, connects an external tubular casing 32 of needle portion 14 to the body 18.

As shown in FIG. 2, needle portion 14 comprises external tubular casing 32 which is closed at a distal end 36 of the casing by a termination 40. Close to termination there is an opening 44 also herein also termed a window 44.

A hollow tubular needle 64 is positioned within casing 32, and the needle is supported within the casing, by a needle holder 112, described below. As is described in more detail below, when vitrector 10 is operative, needle 64 oscillates longitudinally within casing 32, so that a distal tip 128 of the needle traverses window 44. A proximal end 130 of the needle couples to exhaust tubing 138, so that in operation of the vitrector vitreous humor traverses an internal lumen 140 of the needle and is aspirated via exhaust tubing 138.

As stated above, vitrector motor 20 is located within body 18, and a distal section of the motor is shown in FIG. 2. Motor 20 is connected to a shaft coupler 88, which is in turn connected to a drive shaft 92. When operative, motor 20 rotates shaft coupler 88 and drive shaft 92. Shaft 92 is retained by bearings 96 that are located within a housing 98, and the shaft is connected to a cylindrical cam 100, also herein termed a barrel cam 100. There is a circumferential groove 104 in an external radial surface 106 of cam 100 and a cam follower 108, fixedly connected to a needle holder 112 is constrained to travel in the groove. Needle holder 112 fixedly grips needle 64, and couples the needle to cam follower 108.

Holder 112 is itself constrained to only travel in a distal or proximal direction, by being configured to travel on two rods 116—one rod 116 is visible in the cross-section of FIG. 2, and two rods 116 are shown in an inset I1 which is an orthogonal section A—A to drive shaft 92, taken along a line segment 101. To provide the constraint, as shown in inset I1, two cylindrical holes 118, having diameters that match those of rods 116, are formed in holder 112, and the rods act as rails upon which holes 118 slide.

Rods 116 are fixed to housing 98, so that they are parallel to drive shaft 92. Thus, as holder 112 moves, the rods act as guides constraining holder 112 and its connected needle 64 to travel parallel to drive shaft 92. Drive shaft 92 has an axis of symmetry 90 and the drive shaft is attached to cam 100 so that axis of symmetry 90 is collinear with a cam axis 102 of the cam. Axis of symmetry 90 and axis 102 are also collinear with an axis of symmetry 110 of needle 64. Axis of symmetry 110 is also herein termed needle axis 110. It will be understood that the statement above that holder 112 is constrained to only travel distally or proximally is equivalent to stating that the holder is constrained to travel parallel to axis 110.

In order for needle 64 to oscillate distally and proximally as required, i.e., parallel to its axis of symmetry 110, follower 108, to which the needle is fixedly connected, should also move parallel to axis of symmetry 110. As stated above, follower 108 is constrained to travel in groove 104, so that, as cam 100 rotates, the groove is configured to apply the required motion to the follower. It will be understood that if groove 104 lies completely in a single plane normal to needle axis 110, there is no motion of the follower parallel to the axis. Consequently, at least a portion of groove 104 is configured to lie outside any given plane normal to needle axis 110.

The oscillations of needle 64, together with connected holder 112, if unmitigated, generate vibrations and noise. Embodiments of the disclosure reduce the vibrations and noise by adding a countervailing, oscillating, mass 120 to the vitrector, and by arranging that the oscillations of the mass balance those of holder 112 and needle 64, as is described hereinbelow. Mass 120 acts as a counterweight to holder 112 and needle 64, and is herein also termed counterweight 120.

Counterweight 120 is constrained to travel parallel to needle axis 110, by being configured to travel on two rods 124—one is visible in the cross-section of FIG. 2—and two rods 124 are shown in inset I1. Rods 124 are fixed to housing 98, so that they are parallel to needle axis 110. As shown in inset I1, two cylindrical holes 144, having diameters which match those of rods 124, are formed in counterweight 120, and the rods act as rails upon which holes 144 slide.

A second cam to follower 126 is connected counterweight 120, and is constrained to travel in groove 104. Thus, as for needle 64 and holder 112, as cam 100 rotates counterweight 120 also oscillates parallel to needle axis 110.

The combination of motor 20, cam 100, and followers 100 and 126 may be considered to act as a driver 134 for needle 64.

FIG. 3 is a cross-section of selected components of vitrector 10, where for clarity, while some of the components illustrated in FIG. 2 are not shown, components that are shown are drawn on a set of Cartesian axes. In the description herein of components of vitrector 10, the components are assumed to be located on the illustrated set of xyz right orthogonal axes, where the z-axis is collinear with needle axis 110, axis of symmetry 90, and cam axis 102, and the positive direction of the z-axis is the distal direction. The cross-sections of FIGS. 2 and 3 are assumed to be in a yz plane, where the y-axis is normal to the z-axis and is in the plane of the paper. The x-axis is normal to the paper, and the origin of the set of xyz axes is assumed, by way of example, to be on a distal face 114 of cam 100.

FIGS. 4A, 4B are schematic illustrations of cam 100 and its two followers, from different viewpoints. FIG. 4A is a view of cam 100 and the followers as seen looking along an axis of the cam, e.g., along the z-axis. FIG. 4B is a view of the cam and its followers as seen looking from a side of the cam, e.g., along the x-axis. The view in FIG. 4B is taken with the cam rotated so that the followers have their maximum displacement, parallel to the z-axis, from their mean positions.

To balance the oscillations of needle 64, follower 126 and follower 108 are respectively located in two different azimuthal locations in groove 104, and the groove is configured so that as cam 100 rotates the motions of the two followers are always in antiphase. In order that the followers move in antiphase, in an embodiment of the disclosure follower 108, follower 126, and cam axis 102 lie in a common plane 132, herein assumed to be the yz plane, corresponding to the plane of the paper of FIG. 4B. Common plane 132 is also herein termed cam-follower plane 132.

Referring to FIG. 4A, if the azimuthal locations of follower 108 and follower 126, measured with respect to the x-axis, are θ₁ and θ₂ respectively, then an absolute difference of the azimuthal locations is approximately 180°, as shown by equation (1):

$$⌊ {θ}_{2} - {θ}_{1} ⌋ ≅ 180 ° \qquad ( 1 )$$

Referring to FIG. 4B, in addition to the constraint of equation 1, for the followers to move in antiphase throughout the complete period of revolution of cam 100, then the path of groove 104 should have a 2-fold axis of symmetry perpendicular to cam axis 102. In FIG. 4B the 2-fold axis of symmetry of the path followed by groove 104 is perpendicular to cam-follower plane 132.

In a disclosed embodiment, a projection of the path of groove 104, onto cam-follower plane 132 and as illustrated in FIG. 4B, is a sigmoid. In some embodiments the sigmoid has a shape corresponding to a projection of a helix onto cam-follower plane 132. Other shapes of the groove, agreeing with the conditions for the groove described above, will be apparent to those having ordinary skill in the art, and all such shapes are assumed to be comprised within the scope of the present disclosure.

In the following description groove 104 is assumed, by way of example and for simplicity and clarity, to be elliptical and to lie in a plane having a normal that makes a non-zero angle α with cam axis 102. Those having ordinary skill in the art will be able to adapt the description, mutatis mutandis, for shapes other than ellipses, such as those having the sigmoid projections referred to above. A projection 136 of the path of elliptical groove 104 is a straight line that has been drawn in FIG. 4B, and the projection assumes that cam 100 has been rotated so that the major axis of the ellipse lies in cam-follower plane 132. Cam 100 is assumed to have a diameter D.

When motor 20 is powered on, it rotates drive shaft 92 and cylindrical cam 100. The rotation of cam 100 causes groove 104 to rotate around cam axis 102, and this latter rotation causes cam follower 108 to oscillate distally and proximally, parallel to the z-axis and cam axis 102, due to the restraints on the motion of holder 112. A frequency of oscillation, f, depends on the rate of rotation of the motor. A typical value for f is in the approximate range of 500-3000 oscillations per minute. Because follower 108 is fixed to holder 112, the holder and gripped needle 64 also oscillate distally and proximally at frequency f. For the elliptical groove 104 considered herein, a peak-peak amplitude A of oscillation of follower 108, and thus of connected needle 64, measured parallel to cam axis 102, is given by equation (2):

$$A = D \cdot tan α \qquad ( 2 )$$

As motor 20 rotates cam 100, it also causes follower 126 and attached counterweight 120 to oscillate distally and proximately, parallel to cam axis 102, at frequency f. The peak-peak amplitude of oscillation of follower 126 and counterweight 120 is as given by equation 2. However, the oscillation of follower 126 and counterweight 120 is in antiphase to the oscillation of follower 108 and its connected elements.

The antiphase relationship between the positions of the two followers is illustrated by equations (3) and (4):

$${z}_{1} ( t ) = \frac{A}{2} sin 2 π ft \qquad ( 3 )$$

$$and$$

$${z}_{2} ( t ) = \frac{A}{2} sin ( 2 π ft + π ) = - \frac{A}{2} sin 2 π ft \qquad ( 4 )$$

where z₁ (t) and z₂ (t) are respectively positions of followers 108 and 126 at a time t, measured with respect to the z axis, and

A is given by equation (2) (the arguments of the sin function are in radians).

As is described above, by providing counterweight 120 for vitrector 10, vibrations of the vitrector are reduced when the vitrector is operated. To further reduce vibrations, in an embodiment of the disclosure, masses of counterweight 120 and its fixedly connected follower 126 are selected so that a center of mass of the combination of these components, taken together with the masses of follower 108 and its fixedly connected components holder 112 and needle 64, resides on an axis collinear with cam axis 102. In some embodiments a mass M₁ of holder 112 is approximately equal to a mass M₂ of counterweight 120.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values #10% of the recited value, e.g. “about 90%” may refer to the range of values from 81% to 99%.

### EXAMPLES

Example 1. Surgical apparatus, comprising:

a needle (64) configured to be inserted into an organ of a human subject;

a counterweight (120); and

a needle driver (134), comprising:

a cylindrical cam (100) configured to rotate about a cam axis and having a curved circumferential groove (104) extending around a radial surface of the cam;

a motor (20) coupled to rotate the cylindrical cam about the cam axis;

a first follower (108) mounted to travel in the groove at a first azimuthal location and coupled to the needle so as to cause the needle to oscillate at a predefined frequency parallel to the cam axis as the cam rotates; and

a second follower (126) coupled to the counterweight and mounted to travel in the groove at a second azimuthal location selected so as to cause the counterweight to oscillate parallel to the cam axis in antiphase to the needle.

Example 2. The surgical apparatus according to example 1, wherein a path followed by the curved circumferential groove is not completely in a plane normal to the cam axis.

Example 3. The surgical apparatus according to example 1, wherein the first follower, the second follower, and the cam axis reside in a plane.

Example 4. The surgical apparatus according to example 1, wherein a path followed by the curved circumferential groove has a 2-fold axis of symmetry perpendicular to the cam axis.

Example 5. The surgical apparatus according to example 1, wherein a path followed by the curved circumferential groove comprises an ellipse.

Example 6. The surgical apparatus according to example 1, wherein a projection of a path followed by the curved circumferential groove onto a plane containing the cam axis comprises a sigmoid.

Example 7. The surgical apparatus according to example 6, wherein the sigmoid has a shape corresponding to a projection of a helix onto the plane.

Example 8. The surgical apparatus according to example 1, wherein the first azimuthal location and the second azimuthal location differ by a value between 162° and 198°.

Example 9. The surgical apparatus according to example 1, wherein the first follower is coupled to the needle by a holder, and wherein the first follower, the holder and the needle comprise first masses, and wherein the second follower and the counterweight comprise first masses, and wherein a center of mass of the first and second masses resides on an axis collinear with the cam axis.

Example 10. The surgical apparatus according to example 1, wherein the first follower is coupled to the needle by a holder having a first mass M₁, and wherein the counterweight has a second mass M₂ having a value between 0.9·M₁ and 1.1·M₁.

Example 11. A method for producing surgical apparatus, comprising:

configuring a needle (64) to be inserted into an organ of a human subject;

providing a counterweight (120);

configuring a cylindrical cam (100) to rotate about a cam axis, the cam having a curved circumferential groove (104) extending around a radial surface of the cam;

coupling a motor (20) to rotate the cylindrical cam about the cam axis;

mounting a first follower (108) to travel in the groove at a first azimuthal location and coupling the first follower to the needle so as to cause the needle to oscillate at a predefined frequency parallel to the cam axis as the cam rotates; and

coupling the counterweight to a second follower (126) and mounting the second follower to travel in the groove at a second azimuthal location selected so as to cause the counterweight to oscillate parallel to the cam axis in antiphase to the needle.

It will be appreciated that the examples described above are cited by way of example, and that the present disclosure is not limited to what has been particularly shown and described hereinabove. Rather, the scope of the present disclosure includes both combinations and subcombinations of various features described the hereinabove, as well as variations and modifications thereof which would occur to persons skilled in the art upon reading the foregoing description and which are not disclosed in the prior art.

1. Surgical apparatus, comprising:
a needle configured to be inserted into an organ of a human subject;
a counterweight; and
a needle driver, comprising:
a cylindrical cam configured to rotate about a cam axis and having a curved circumferential groove extending around a radial surface of the cam;
a motor coupled to rotate the cylindrical cam about the cam axis;
a first follower mounted to travel in the groove at a first azimuthal location and coupled to the needle so as to cause the needle to oscillate at a predefined frequency parallel to the cam axis as the cam rotates; and
a second follower coupled to the counterweight and mounted to travel in the groove at a second azimuthal location selected so as to cause the counterweight to oscillate parallel to the cam axis in antiphase to the needle.

2. The surgical apparatus according to claim 1, wherein a path followed by the curved circumferential groove is not completely in a plane normal to the cam axis.

3. The surgical apparatus according to claim 1, wherein the first follower, the second follower, and the cam axis reside in a plane.

4. The surgical apparatus according to claim 1, wherein a path followed by the curved circumferential groove has a 2-fold axis of symmetry perpendicular to the cam axis.

5. The surgical apparatus according to claim 1, wherein a path followed by the curved circumferential groove comprises an ellipse.

6. The surgical apparatus according to claim 1, wherein a projection of a path followed by the curved circumferential groove onto a plane containing the cam axis comprises a sigmoid.

7. The surgical apparatus according to claim 6, wherein the sigmoid has a shape corresponding to a projection of a helix onto the plane.

8. The surgical apparatus according to claim 1, wherein the first azimuthal location and the second azimuthal location differ by a value between 162° and 198°.

9. The surgical apparatus according to claim 1, wherein the first follower is coupled to the needle by a holder, and wherein the first follower, the holder and the needle comprise first masses, and wherein the second follower and the counterweight comprise first masses, and wherein a center of mass of the first and second masses resides on an axis collinear with the cam axis.

10. The surgical apparatus according to claim 1, wherein the first follower is coupled to the needle by a holder having a first mass M₁, and wherein the counterweight has a second mass M₂ having a value between 0.9·M₁ and 1.1·M₁.

11. A method for producing surgical apparatus, comprising:
configuring a needle to be inserted into an organ of a human subject;
providing a counterweight;
configuring a cylindrical cam to rotate about a cam axis, the cam having a curved circumferential groove extending around a radial surface of the cam;
coupling a motor to rotate the cylindrical cam about the cam axis;
mounting a first follower to travel in the groove at a first azimuthal location and coupling the first follower to the needle so as to cause the needle to oscillate at a predefined frequency parallel to the cam axis as the cam rotates; and
coupling the counterweight to a second follower and mounting the second follower to travel in the groove at a second azimuthal location selected so as to cause the counterweight to oscillate parallel to the cam axis in antiphase to the needle.

12. The method according to claim 11, wherein a path followed by the curved circumferential groove is not completely in a plane normal to the cam axis.

13. The method according to claim 11, wherein the first follower, the second follower, and the cam axis reside in a plane.

14. The method according to claim 11, wherein a path followed by the curved circumferential groove has a 2-fold axis of symmetry perpendicular to the cam axis.

15. The method according to claim 11, wherein a path followed by the curved circumferential groove comprises an ellipse.

16. The method according to claim 11, wherein a projection of a path followed by the curved circumferential groove onto a plane containing the cam axis comprises a sigmoid.

17. The method according to claim 16, wherein the sigmoid has a shape corresponding to a projection of a helix onto the plane.

18. The method according to claim 11, wherein the first azimuthal location and the second azimuthal location differ by a value between 162° and 198°.

19. The method according to claim 11, wherein the first follower is coupled to the needle by a holder, and wherein the first follower, the holder and the needle comprise first masses, and wherein the second follower and the counterweight comprise first masses, and wherein a center of mass of the first and second masses resides on an axis collinear with the cam axis.

20. The method according to claim 11, wherein the first follower is coupled to the needle by a holder having a first mass M₁, and wherein the counterweight has a second mass M₂ having a value between 0.9·M₁ and 1.1·M₁.
